(19) United States
(12) Patent Application Publication
(10) Pub. No.: US 2013/0261545 A1
(43) Pub. Date: Oct. 3, 2013

(54) DRUG ELUTING FOLDING BALLOON

(71) Applicant: Thomas P. Osypka, Palm Harbor, FL (US)

(72) Inventor: Thomas P. Osypka, Palm Harbor, FL (US)

(73) Assignee: Oscor Inc., Palm Harbor, FL (US)

(21) Appl. No.: 13/905,608

(22) Filed: May 30, 2013

Related U.S. Application Data
(63) Continuation-in-part of application No. 13/343,228, filed on Jan. 4, 2012.
(60) Provisional application No. 61/460,525, filed on Jan. 4, 2011.

(51) Int. Cl. A61M 25/10

(52) U.S. Cl. CPC A61M 25/10
USPC 604/103.02

(57) ABSTRACT

A balloon catheter includes a first outer tubular member, a second inner tubular member, an inflatable balloon, and a drug coating applied to at least a portion of the inflatable balloon. The inflatable balloon has opposing distal and proximal end portions. The inflatable balloon resides on the first and second tubular members in a twisted turn orientation of a pre-determined angle relative to the proximal end portion of the inflatable balloon attached to the first outer tubular member, such that the second inner tubular member achieves a constant torque of the balloon to twist and fold itself when retracted back into a portion of the first outer tubular member. Folds caused by the twisted turn orientation are configured and adapted to protect the drug coating while the inflatable balloon is deflated, thereby reducing premature drug elution and increasing the reliability and consistency of drug delivery.

### CROSS REFERENCE TO RELATED APPLICATIONS

The present application is a continuation-in-part of co-pending U.S. application Ser. No. 13/343,228 filed Jan. 4, 2012, which is incorporated herein by reference in its entirety.

### BACKGROUND OF THE INVENTION

1. Field of the Invention

The present invention generally relates to balloon catheters, and more particularly to a drug eluting medical balloon catheter device and method for protecting a drug and delivering the drug to a surgical site using a balloon catheter that folds onto a catheter tube during balloon deflation, the folds protecting the drug, thereby reducing premature drug elution.

2. Description of Related Art

A balloon catheter generally comprises an inflatable balloon that is mounted along a distal end of an elongate catheter body (i.e., shaft). Balloon catheters are used by physicians in a wide variety of therapeutic procedures. In one common use, a folded balloon catheter is advanced through a blood vessel to a region that has become occluded by atherosclerotic plaque. The balloon is inflated to dilate the occluded region and thereby improve the flow of blood through the vessel. In another common use, an expandable stent is provided along the exterior of the balloon. The balloon is advanced to the treatment site and is then inflated to deploy the stent. The balloon is then deflated and the balloon catheter is withdrawn from the patient. The expanded stent remains in the blood vessel to provide support to the vessel wall. In another common use, a drug is applied to the balloon surface for drug delivery to a vessel. The drug is applied to the balloon surface prior to insertion of the balloon catheter

Balloon catheters are typically formed of a very thin, yet strong material. During manufacture, a balloon is folded at a number of locations along its longitudinal axis. After the balloon is folded in a variety of locations, the folds are wrapped around the catheter to reduce the balloon to a constrained condition having a very small diameter. In the case wherein the balloon is used to deploy a stent, the stent is crimped onto the balloon after the folding step. The balloon is advanced through the blood vessel to a treatment site while in the constrained condition. The balloon is typically inflated by directing a fluid through a lumen in the catheter to pressurize the balloon. During inflation, the balloon unfolds, rather than stretches.

It is to be appreciated that arterial blockages are typically caused by the buildup of plaque in the arteries of a patient often having severe consequences. This is because the buildup of plaque in arteries reduces, and eventually, blocks blood flow through the affected vessel. When blood flow is reduced in a coronary artery, the heart muscle becomes deprived of oxygen, and the patient is prone to suffer angina. In severe cases of coronary artery blockage, the patient suffers a heart attack.

Many modern surgical techniques have been developed to alleviate the stenoses that are formed when plaque builds up in a patient's arteries. For example, a large number of balloon angioplasty devices exist for relieving arterial stenoses by compression of the stenosis. In several respects, balloon angioplasty devices afford numerous advantages over alternative methods. Foremost among these advantages is that open heart bypass surgery can often be avoided by using angioplasty surgical techniques to relieve stenoses in the arteries that supply blood to the heart. For obvious reasons, it is preferable to avoid open heart surgery when possible because such surgery, as is well known, is invasive and typically requires a significant post-operative recovery time. Accordingly, it is preferable to use relatively simpler angioplasty surgical procedures when such procedures are feasible. Importantly, angioplasty procedures are efficacious in the peripheral arteries as well as in the arteries that supply blood to the heart.

In angioplasty surgery, the balloon of a balloon catheter is initially attached to a catheter tube in a deflated configuration, with the catheter tube connecting a fluid source in fluid communication with the balloon. The balloon is then positioned at the desired location in the affected artery by advancing the catheter through the artery until the balloon is positioned across a stenosis that is to be treated. Once the balloon has been properly positioned, fluid is infused into the balloon. As the balloon expands, it dilates the lumen of the artery and compresses the plaque which may then break up or flatten out against the arterial wall. The balloon is then deflated and, once in its deflated configuration, it is either withdrawn from the artery or placed across another stenosis, to restore normal blood flow through the artery.

Balloon catheters are also typically used for internal drug delivery in angioplasty surgery, and other treatments of arteries and vessels. Generally, a drug is embedded in a coating on the balloon surface and the balloon catheter is used to deliver the drug to a targeted area in a vessel, artery, or the like. However, a noted problem associated with balloon catheters exists during the insertion and positioning of the balloon into the vessel. Specifically, it is desirable that the drug coating is maintained during the insertion and positioning of the balloon to the targeted area in the vessel and eluted when the balloon reaches the desired location. If the drug coating gets prematurely eluted, diluted or absorbed during the insertion and positioning of the balloon, inconsistencies in treatment can occur.

Accordingly, it is an object of the present invention to provide a balloon catheter having a mechanism, which causes the balloon to consistently deliver a drug to a targeted area in a vessel, artery, or the like while reducing dilution, washing away and premature absorption. Another object of the present invention is to provide a device which is relatively simple to manufacture, easy to use, and comparatively cost effective.

### SUMMARY OF THE INVENTION

The subject invention is directed to a new and useful drug eluting medical balloon catheter. The balloon catheter includes a first outer tubular member, a second inner tubular member, an inflatable balloon, and a drug coating applied to at least a portion of the inflatable balloon. The second inner tubular member is configured for slideable reception within the first outer tubular member such that a distal end portion of the second inner tubular member extends distally from a distal end portion of the first outer tubular member. The inflatable balloon has opposing distal and proximal end portions.

The proximal end portion of the inflatable balloon is affixed to the distal end portion of the first outer tubular member and the distal end portion of the inflatable balloon is affixed to the distal end portion of the second inner tubular member. The inflatable balloon resides on the first and second tubular members in a twisted turn orientation of a pre-determined angle relative to the proximal end portion of the inflatable balloon attached to the first outer tubular member. Folds caused by the twisted turn orientation are configured and adapted to protect the drug coating while the inflatable balloon is deflated, thereby reducing premature drug elution and increasing the reliability and consistency of drug delivery.

In one embodiment of the subject invention, the inflatable balloon can be fabricated from rigid plastic and/or the balloon can be configured and adapted to be inflated with liquid. The inflatable balloon can also be configured and adapted to return to the twisted turn orientation relative to the outer tubular member when the inflatable balloon is caused to be deflated. The twisted turn orientation can be at least forty-five degrees. In addition, the twisted turn orientation can be configured such that the second inner tubular member achieves a constant torque of the inflatable balloon to twist and fold itself when retracted back into a portion of the first outer tubular member. The balloon catheter can be configured and adapted to be inserted within an introducer sheet lumen. In addition, the drug coating can be a polymer drug coating.

In another aspect, a tubular assembly consists of an inner tubular member configured for slideable reception within an outer tubular member, a balloon member, and a drug coating applied to at least a portion of the balloon member. A distal end portion of the inner tubular member extends distally from a distal end portion of the outer tubular member. Balloon member has first and second end regions wherein a first end region is attached to the distal end portion of the inner tubular member and the second end region is attached to the distal end portion of the outer tubular member. Balloon member is configured and adapted to fold when deflated and protect the drug coating therein reducing premature drug elution and increasing the reliability and consistency of drug delivery.

In certain embodiments, the balloon member can reside on the tubular assembly with a twisted turn orientation, much as described above. The balloon member can be fabricated from rigid plastic, configured to return to the twisted turn orientation relative to the outer tubular member when the balloon member is caused to be deflated, and/or configured to be inflated with liquid. In addition, folds, much as described above, can be caused by the twisted turn orientation of the balloon member. The inner tubular member can achieve a constant torque of the balloon member to twist and fold itself when retracted back into a portion of the outer tubular member. In addition, the tubular assembly can be configured and adapted to be inserted within an introducer sheet lumen.

In yet another aspect, a method of protecting a drug on a balloon catheter comprises the steps of providing an introducer sheet defining an inner lumen, providing a tubular assembly with a balloon member, slideably receiving the tubular assembly within the inner lumen of the introducer sheet, positioning the introducer sheet at the surgical site, advancing the tubular assembly from a distal end of the introducer sheet, inflating the balloon member at the surgical site, deflating the balloon member at the surgical site, and retracting the tubular assembly into the inner lumen of the introducer at the distal end of the introducer sheet.

The tubular assembly consists of an inner tubular member configured for slideable reception within an outer tubular member such that a distal end portion of the inner tubular member extends distally from a distal end portion of the outer tubular member. A first end region of the balloon member is attached to the distal end portion of the inner tubular member and a second end region of the balloon member is attached to the distal end portion of the outer tubular member. Much as described above, the balloon member resides on the tubular assembly with a twisted turn orientation and at least a portion of the balloon member is coated with a drug. The tubular assembly is advanced such that the balloon member is exposed from the inner lumen of the introducer sheet. The inflating of balloon member exposes the drug and allows for delivery of the drug to the surgical site. The tubular assembly is retracted such that a substantial portion of the balloon member is located within the inner lumen of the introducer sheet.

In certain embodiments, when the balloon member is caused to inflate it can un-twist itself relative to the distal end portion of the outer tubular member. The twisted turn orientation can be much as described above. When the balloon member is caused to deflate, it can return to the said twisted orientation relative to the distal end portion of the outer tubular member when retracted back into a portion of the outer tubular member. In addition, the inner tubular member can achieve a constant torque of the balloon member to twist and fold itself when retracted back into a portion of the outer tubular member. The balloon member can be fabricated from rigid plastic and/or configured and adapted to be inflated with liquid.

These and other features of the drug eluting folding balloon of the subject invention and the manner in which it is employed will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the subject invention appertains will readily understand how to make and use the drug eluting folding balloon subject invention without undue experimentation, preferred embodiments thereof will be described in detail herein below with reference to certain figures, wherein:

FIG. 1 illustrates a foldable balloon catheter in accordance with the present invention, showing the foldable balloon;

FIG. 2 illustrates the foldable balloon catheter of FIG. 1, showing the outer shaft tubing;

FIG. 3 illustrates the process for folding the foldable balloon of FIG. 1 in accordance with the present invention;

FIG. 4 illustrates the foldable balloon catheter of FIG. 1, showing the foldable balloon in a folded position on outer shaft tubing ready for insertion into a lumen;

FIG. 5 illustrates the foldable balloon catheter of FIG. 1, showing the foldable balloon in at least a partially inflated position;

FIG. 6 illustrates the foldable balloon catheter of FIG. 1, showing the foldable balloon in a fully wrapped position;

FIG. 7 illustrates the foldable balloon catheter of FIG. 1 being introduced into a lumen of an introducer sheath;

FIGS. 8A-C illustrate foldable balloon catheter of FIG. 1 being slideably received within the lumen of the introducer sheath;

FIGS. 9A-C illustrate foldable balloon catheter of FIG. 1, showing the sequential steps of the foldable balloon being inflated;

FIG. 10 illustrates the foldable balloon catheter of FIG. 1, showing the foldable balloon in a fully inflated position;

FIG. 11 illustrates the foldable balloon catheter of FIG. 1, showing the foldable balloon being deflated;

FIG. 12 illustrates the foldable balloon catheter of FIG. 1, showing the foldable balloon in a fully deflated and tightly wrapped position;

FIG. 13 illustrates the foldable balloon catheter of FIG. 1 being retracted back into the lumen of the introducer sheath;

FIG. 14 illustrates a drug eluting foldable balloon catheter in accordance with the present invention, showing a foldable balloon and a drug coating on the balloon;

FIG. 15 illustrates the drug eluting foldable balloon catheter of FIG. 14, showing the foldable balloon being folded about outer shaft tubing;

FIG. 16 illustrates the drug eluting foldable balloon catheter of FIG. 14, showing the drug eluting foldable balloon catheter at a surgical site, with the foldable balloon in a fully deflated and tightly wrapped position, and the drug coating at least partially intact; and

FIG. 17 illustrates the drug eluting foldable balloon catheter of FIG. 14, showing the foldable balloon in a fully inflated position.

### DETAILED DESCRIPTION OF PREFERRED EMBODIMENTS

The present invention is now described more fully with reference to the accompanying drawings, in which an illustrated embodiment of the present invention is shown. The present invention is not limited in any way to the illustrated embodiment as the illustrated embodiment described below is merely exemplary of the invention, which can be embodied in various forms, as appreciated by one skilled in the art. Therefore, it is to be understood that any structural and functional details disclosed herein are not to be interpreted as limiting, but merely as a basis for the claims and as a representative for teaching one skilled in the art to variously employ the present invention. Furthermore, the terms and phrases used herein are not intended to be limiting but rather to provide an understandable description of the invention.

It is to be appreciated and understood that the present invention, in accordance with the illustrated embodiments, is directed to a system and method for protecting a drug and delivering a drug to a surgical site using foldable balloon catheters, wherein the balloon is folded onto itself tightly around the catheter in a twisted (overlapping) matter each time the balloon is caused to be deflated. The folds of the balloon protect the drug during insertion and positioning, therein reducing premature drug elution and providing reliable and consistent delivery of the drug to the surgical site.

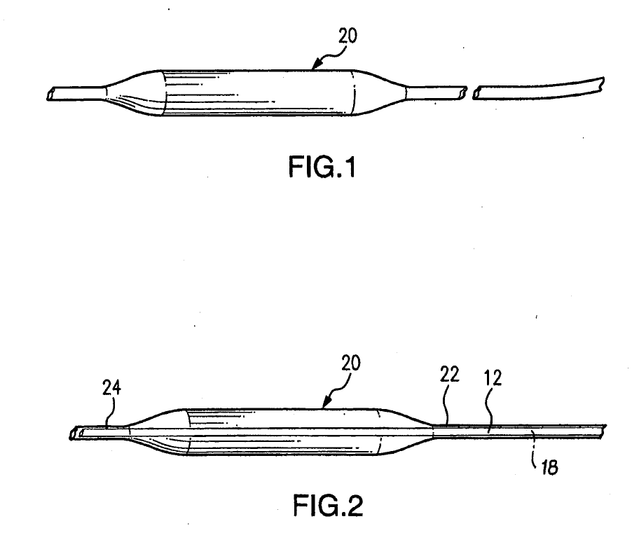

As will be apparent from the below description in accordance with the illustrated embodiments of FIGS. 1 to 17, the balloon catheter 10 in accordance with the illustrated embodiments of the invention includes an outer shaft tubing 12 and a distal inner shaft tubing 14 (e.g., a guidewire lumen—as the inner shaft lumen is often used as a guidewire lumen, whereby the balloon catheter is introduced over a guidewire into the vessel to the desired location) and a balloon 20 affixed thereto as described below. It is to be appreciated the inner shaft tubing 14 resides the length of the catheter 10 and is slideably received within the outer tubing 12 having a distal end portion 16 extending distally from a distal end portion 18 of the outer tubing 12. A proximal section 22 of a balloon 20 is mounted onto the outer tubing 12 by known adhesive means (e.g., heat, glue, and the like) with a distal section 24 of the balloon 20 being mounted onto the inner tubing 14 also by known adhesive means (e.g., heat, glue, and the like) such that the balloon 20 is sealed between both the inner 14 and outer tubing 12 members in an manner suitable for inflation. Further, a self twisting and self wrapping of the balloon 20 during inflation and deflation is achieved whereby the distal end 24 of the balloon 20 is prescribed with an at least forty-five degree twisted turn orientation relative to the proximal section 22 of the balloon 20 which is then attached to the distal inner tubing 14 in a twisted condition such that the inner tubing 14 achieves a constant torque of the balloon 20 to twist and fold itself. Depending on the shape and size of the balloon, several twists exceeding 360° may be provided. A drug coating 26 is applied to at least a portion of balloon 20, wherein folds 28 caused by the twisted turn orientation are configured and adapted to protect drug coating 26 while balloon 20 is deflated, thereby reducing premature drug elution during insertion and delivery of balloon 20 to the surgical site.

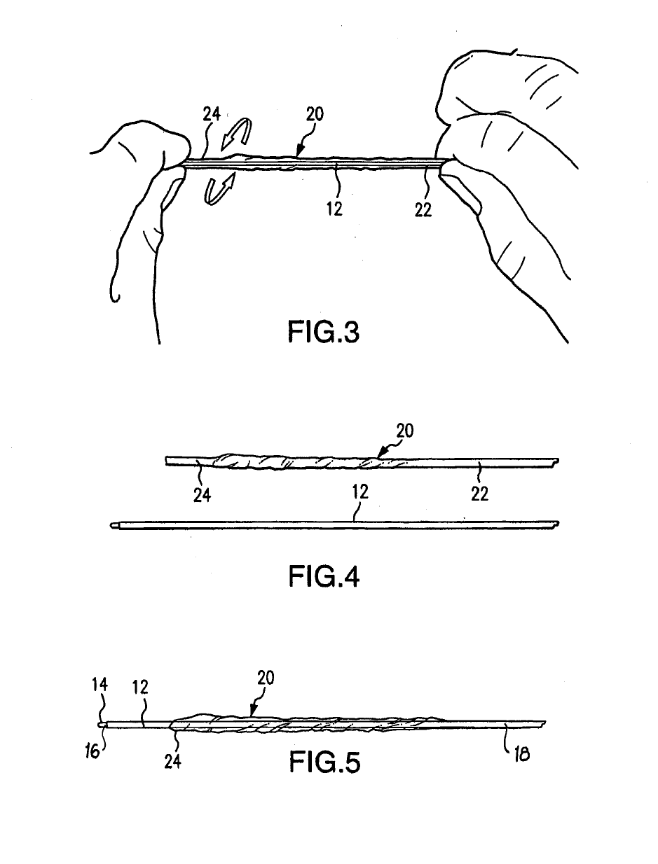

Turning now descriptively to the drawings, in which similar reference characters denote similar elements throughout the several views, FIG. 1 depicts a balloon 20 for use with a catheter device 10. Preferably medical grade material is used to fabricate balloon 20 which material is typically very thin but rather rigid plastic, so that the inflated diameter is predictable and doesn't vary greatly as a function of inflation pressure. Because of this, catheter balloons do not stretch like a rubber balloon when inflated, but rather they unfold. FIG. 2 depicts an outer shaft tubing member 12 (having a inner shaft tubing member 14 slideably received therewithin) received through the interior portion of the balloon 20 so as to extend from the proximal section 22 of balloon 20 to distal end section 24. FIG. 3 depicts the folding of balloon 20 about outer shaft tubing 12. It is to be appreciated that folding is the process of wrapping the wing portions of balloon 20 spirally around the outer shaft tubing 12. Folding may be done by either hand or machine.

When performed by hand as shown in FIG. 3, it is accomplished by holding the shaft 12 in one hand while gripping and turning the adjacent part of the balloon 20 around the catheter axis with the other hand. The balloon 20 is preferably folded incrementally, moving both the folding and grasping hands incrementally in steps from the distal 24 to the proximal 22 end of the balloon 20. Following the folding, the balloon 20 is preferably placed in an introducer sheath to hold it in the folded position and guide it to a desired surgical location. FIG. 4 depicts the balloon 20 in a folded position on outer shaft tubing 12 ready for insertion into the lumen 102, shown in FIG. 7, of an introducer 100 while FIG. 5 depicts the balloon 20 in at least a partially inflated position.

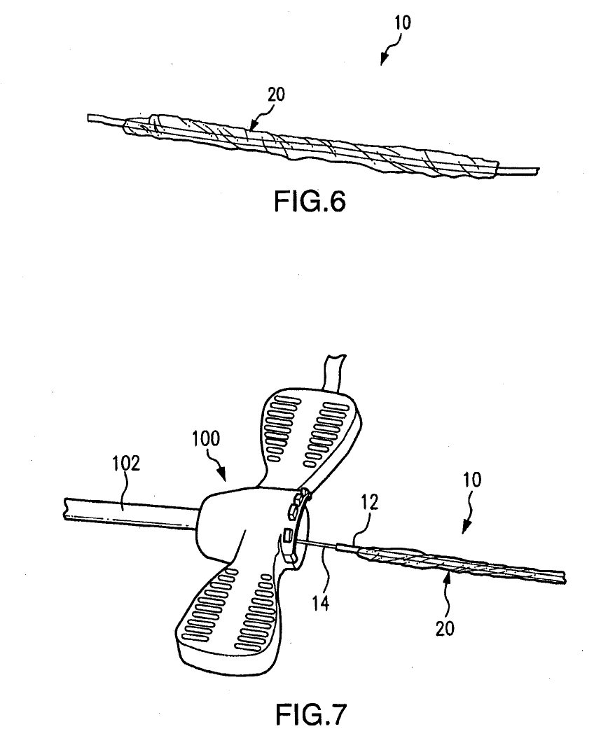

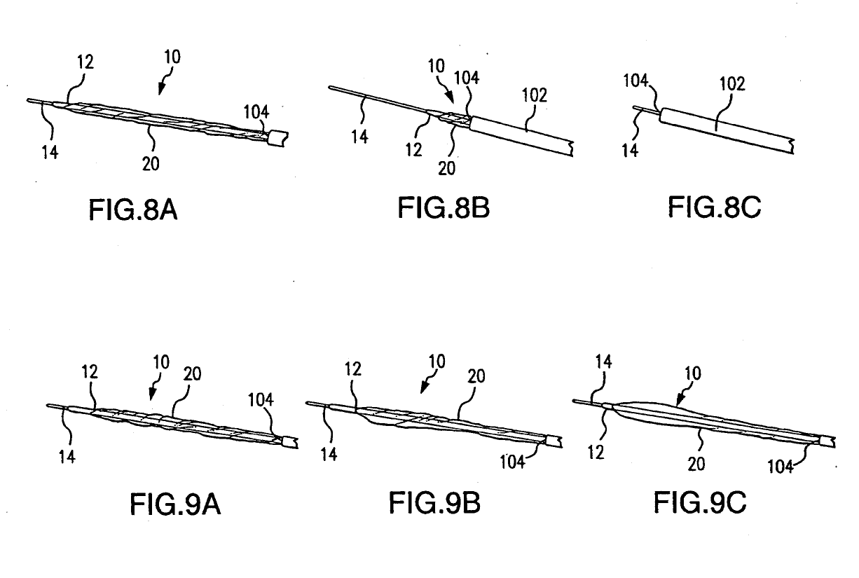

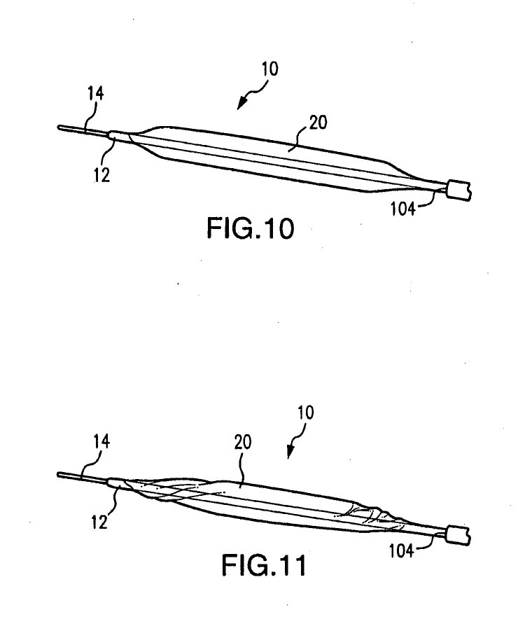

Reference is now made to FIGS. 6 to 13 illustrating the self-twisting balloon features of the present invention. With reference to FIG. 6, illustrated is a balloon 20 of balloon catheter 10 in a fully wrapped position, preferably in preparation for insertion into the lumen 102, shown in FIG. 7, of an introducer sheath 100. FIG. 7 illustrates the balloon catheter 10 (in which the balloon 20 is tightly wrapped around the outer shaft tubing 12 of catheter 10) being introduced into the lumen 102 of introducer sheath 100. FIGS. 8A to 8C illustrate the sequential steps of the balloon catheter 10 (of FIG. 6) being slideably received within the lumen 102 of introducer sheath 100 such that it extends distally from the distal end 104 of the lumen 102 (which is to be understood to be inserted in a vessel) of introducer 100 so as to be exposed to a vessel (FIG. 8C). FIGS. 9A to 9C illustrate the sequential steps of balloon 20 being inflated (via preferably an inflation fluid, such as saline solution or a contrast medium) such that the portion of the balloon 20 extending from the distal end 104 of the lumen sheath 102, unwraps (e.g., unfolds) from the outer shaft tubing 12 of catheter 10. FIG. 10 depicts the balloon 20 in a fully inflated position while FIG. 11 depicts the balloon 20 being deflated, preferably via the use of a pulling vacuum provided on an inflation syringe wherein it is to be appreciated and understood, in accordance with the present invention, the balloon wraps (folds) itself into the wrapped position shown in FIGS. 3A to 3C.

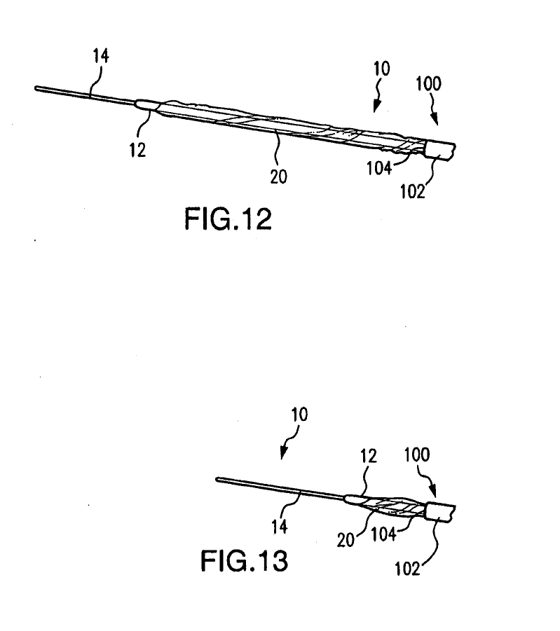

With reference to FIG. 12, the balloon 20 is illustrated in a fully deflated and tightly wrapped position about the outer shaft tubing 12 of catheter 10. As illustrated in FIG. 13, this enables the balloon catheter 12 to be retracted back into the lumen 102 of introducer 100 such that the balloon catheter 10 may be transported to another surgical location or removed from the patient's body via introducer 100.

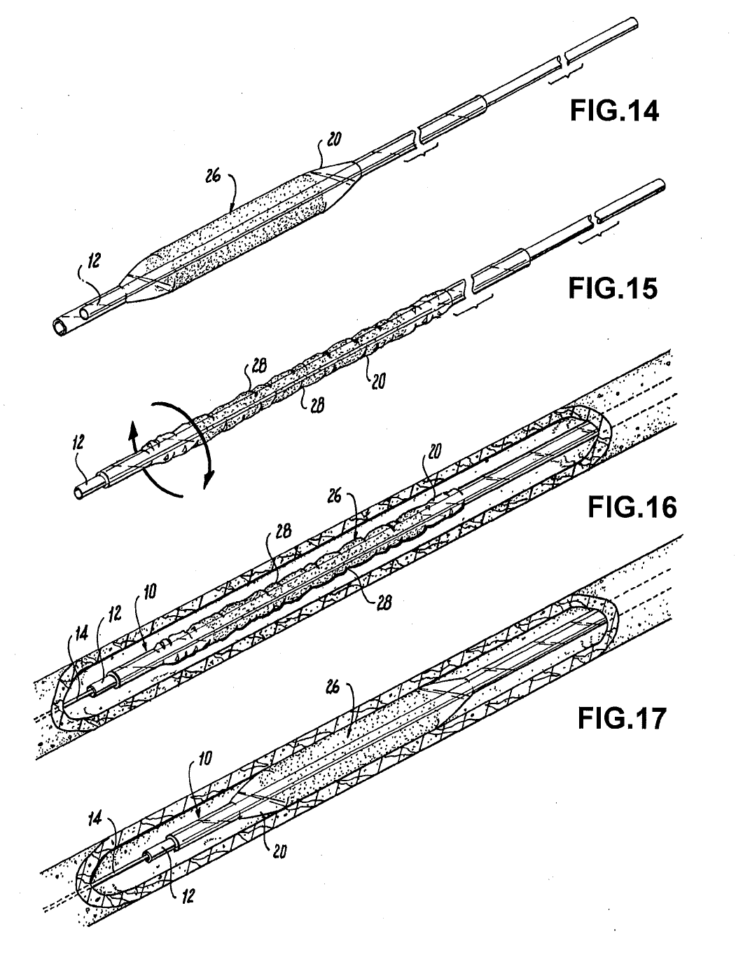

Turning now to FIGS. 14-17, illustrated is a drug eluting foldable balloon catheter in accordance with the present invention, and a method of using the drug eluting foldable balloon catheter. With reference to FIG. 14, balloon 20 for use with catheter device 10, as described above with reference to FIG. 1, is shown. Drug coating 26, is applied to balloon 20. Those skilled in the art will readily appreciate that drug coating 26 can be applied by means of a polymer or the like. Those skilled in the art will also readily appreciate that a variety of drug types can be used for drug coating 26, such as for example, steroids, anti-clotting, anti-thrombolytic, anti-inflammatory, Paclitaxel and Everolimus or the like.

FIG. 15 depicts the folding of balloon 20, as previously described above with reference to FIG. 3. FIG. 15 depicts balloon 20 folding about outer shaft tubing 12. It is to be appreciated folding is the process of wrapping the wing portions of balloon 20 spirally around the outer shaft tubing 12. Folding may be done by either hand or machine, as described above. When deflated, folds 28 are created by folding or wrapping the wing portions of balloon 20. Folds 28 are configured and adapted to protect at least a portion of the previously applied drug coating 26 from prematurely eluting from balloon 20, which can cause dilution, premature absorption, or the like. In addition, by reducing premature elution, the drug can be delivered to the desired surgical site in a more reliable and predictable manner.

Now with reference to FIG. 16, after the folding of balloon 20 and insertion of catheter 10 into lumen 102 of introducer sheath 100, catheter 10 is delivered to the surgical site, in this case a vessel, with drug coating 26 at least partially intact. Lumen 102 and introducer sheath 100 are not shown in FIG. 16, but described above with reference to FIGS. 6-8C. FIG. 17 shows balloon 20 inflated as described above with reference to FIGS. 9A-9C, such that the portion of balloon 20 extending from distal end 104 of lumen sheath 102, has unwrapped (e.g., unfolded) from the outer shaft tubing 12 of catheter 10. When balloon 20 is fully inflated, folds 28 are no longer present to protect drug coating 26, therefore drug coating 26 is permitted to be eluted from balloon 20.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

The above presents a description of a best mode contemplated for carrying out the present balloon folding and wrapping devices and methods, and of the manner and process of making and using them, in such full, clear, concise, and exact terms as to enable any person skilled in the art to which it pertains to make and use these devices and methods. These balloon folding and wrapping devices and methods are, however, susceptible to modifications and alternative method steps from those discussed above that are fully equivalent. Consequently, these balloon folding and wrapping devices and methods are not limited to the particular embodiments disclosed. On the contrary, these balloon folding and wrapping devices and methods cover all modifications and alternative constructions and methods coming within the spirit and scope of the present invention. For instance, a device using a single outer tubing and employing two discrete lumens may also be used in addition to what is illustrated.

The descriptions above and the accompanying drawings should be interpreted in the illustrative and not the, limited sense. While the invention has been disclosed in connection with the preferred embodiment or embodiments thereof, it should be understood that there may be other embodiments which fall within the scope of the invention as defined by the following claims. Where a claim, if any, is expressed as a means or step for performing a specified function, it is intended that such claim be construed to cover the corresponding structure, material, or acts described in the specification and equivalents thereof, including both structural equivalents and equivalent structures, material-based equivalents and equivalent materials, and act-based equivalents and equivalent acts.

What is claimed is:

1. A balloon catheter comprising:
a) a first outer tubular member;
b) a second inner tubular member configured for slideable reception within the first outer tubular member such that a distal end portion of the second inner tubular member extends distally from a distal end portion of the first outer tubular member;
c) an inflatable balloon having opposing distal and proximal end portions wherein the proximal end portion of the inflatable balloon is affixed to the distal end portion of the first outer tubular member and the distal end portion of the inflatable balloon is affixed to the distal end portion of the second inner tubular member wherein the inflatable balloon resides on the first and second tubular members in a twisted turn orientation of a pre-determined angle relative to the proximal end portion of the inflatable balloon attached to the first outer tubular member; and
d) a drug coating applied to at least a portion of the inflatable balloon, wherein folds caused by the twisted turn orientation are configured and adapted to protect the drug coating while the inflatable balloon is deflated, thereby reducing premature drug elution and increasing the reliability and consistency of drug delivery.

2. The balloon catheter as recited in claim 1, wherein the inflatable balloon is fabricated from rigid plastic.

3. The balloon catheter as recited in claim 1, wherein the balloon catheter is configured and adapted to be inserted within an introducer sheet lumen.

4. The balloon catheter as recited in claim 1, wherein the inflatable balloon is configured and adapted to be inflated with liquid.

5. The balloon catheter as recited in claim 1, wherein the inflatable balloon is configured and adapted to return to the twisted turn orientation relative to the first outer tubular member when the inflatable balloon is caused to be deflated.

6. The balloon catheter as recited in claim 1, wherein the twisted turn orientation is at least forty-five degrees.

7. The balloon catheter as recited in claim 1, wherein the twisted turn orientation is configured such that the second inner tubular member achieves a constant torque of the inflatable balloon to twist and fold itself when retracted back into a portion of the first outer tubular member.

8. The balloon catheter as recited in claim 1, wherein the drug coating is a polymer drug coating.

9. A catheter assembly comprising:
a) a tubular assembly consisting of an inner tubular member configured for slideable reception within an outer tubular member such that a distal end portion of the inner tubular member extends distally from a distal end portion of the outer tubular member;
b) a balloon member having first and second end regions wherein a first end region is attached to the distal end portion of the inner tubular member and the second end region is attached to the distal end portion of the outer tubular member; and
c) a drug coating applied to at least a portion of the balloon member, wherein the balloon member is configured and adapted to fold when deflated and protect the drug coating therein reducing premature drug elution and increasing the reliability and consistency of drug delivery.

10. The catheter assembly as recited in claim 9, wherein the balloon member resides on the tubular assembly with a twisted turn orientation of a pre-determined angle relative to the second end region of the balloon member attached to the distal end portion of the outer tubular member.

11. The catheter assembly as recited in claim 10, wherein the twisted turn orientation is at least forty-five degrees.

12. The catheter assembly as recited in claim 10, wherein the inner tubular member achieves a constant torque of the balloon member to twist and fold itself when retracted back into a portion of the outer tubular member.

13. The catheter assembly as recited in claim 9, wherein the balloon member is fabricated from rigid plastic.

14. The catheter assembly as recited in claim 9, wherein the tubular assembly is configured and adapted to be inserted within an introducer sheet lumen.

15. The catheter assembly as recited in claim 9, wherein the balloon member is configured and adapted to be inflated with liquid.

16. The catheter assembly as recited in claim 9, wherein the balloon member is configured and adapted to return to the twisted turn orientation relative to the outer tubular member when the balloon member is caused to be deflated.
